(19) United States
(12) Patent Application Publication
(10) Pub. No.: US 2026/0248791 A1
(43) Pub. Date: Aug. 27, 2026

(54) METHODS FOR TREATING DERMATOMYOSITIS WITH BREPOCITINIB

(71) Applicant: Priovant Therapeutics, Inc., Durham, NC (US)

(72) Inventors: Brendan JOHNSON, New York, NY (US); Paul N. Mudd, JR., New York, NY (US); Matthew Anthony Franco, New York, NY (US); Nikhil Khetarpal, New York, NY (US)

(21) Appl. No.: 19/489,735

(22) PCT Filed: May 21, 2024
(86) PCT No.: PCT/US2024/030335

§ 371 Date: Dec. 3, 2025

Related U.S. Application Data
(60) Provisional application No. 63/506,265, filed on Jun. 5, 2023.
(60) Provisional application No. 63/603,280, filed on Nov. 28, 2023.
(60) Provisional application No. 63/574,382, filed on Apr. 4, 2024.

(51) Int. Cl. A61K 31/506; A61K 9/00; A61P 37/06

(52) U.S. Cl. CPC A61K 31/506; A61K 9/0053; A61P 37/06

(57) ABSTRACT

The present disclosure provides methods related to treatment of a chronic immune-mediated disease of the skin and muscles. In particular, the present disclosure provides methods for the oral administration of a JAK inhibitor, or a pharmaceutically acceptable salt thereof for the treatment of dermatomyositis.

### RELATED APPLICATION INFORMATION

This application claims priority to U.S. Provisional Application No. 63/574,382, filed on Apr. 4, 2024, U.S. Provisional Application No. 63/603,280 filed on Nov. 28, 2023, and U.S. Provisional Application No. 63/506,265 filed on Jun. 5, 2023, 63/506,265 filed on Jun. 5, 2023, the contents of which are herein incorporated by reference.

### FIELD

The present disclosure provides methods related to treatment of a chronic immune-mediated disease of the skin and muscles. In particular, the present disclosure provides methods for the oral administration of a JAK inhibitor, or a pharmaceutically acceptable salt thereof for the treatment of dermatomyositis.

### BACKGROUND

Dermatomyositis (DM) is a rare, debilitating, multi-system idiopathic inflammatory myopathy characterized by skin rash, perifascicular atrophy of muscle fibers and subsequent muscle weakness. In the United States, DM affects approximately 1 in 10 individuals per one million people, irrespective of race and ethnicity. Pathogenetically, DM is linked to the dysregulation of type 1 interferon (IFN-1) pathways. Additionally, key cytokines like IFN-γ, Interleukin (IL)-12, and IL-23 have been implicated in the pathogenesis of DM, substantially contributing to the manifestations across various organ systems, including the skin and muscles. Many treatments for DM involve immunosuppressive medications that can help manage symptoms but do not directly address the underlying disease pathology, presenting certain challenges. These challenges include but are not limited to increased risk of infections due to a weakened immune system and recurrent flares which can lead to a dependency on the use of corticosteroids. Likewise, these drugs can also lead to various side effects and long-term concerns, especially when used at high doses or for extended periods. To enhance outcomes in DM, it is crucial to pursue future treatments that offer improved efficacy, reduced dependency, lower dosage requirements, and compatibility with concurrent therapies, thereby reducing the need for concomitant corticosteroids.

### SUMMARY

Embodiments of the present disclosure include a method for comprising administering to a subject in need of treatment thereof an effective amount [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone (Brepocitinib)

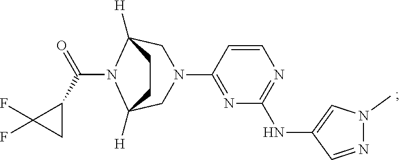 [SMILES: Cn1cc(Nc2nccc(N3C[C@H]4CC[C@@H](C3)N4C(=O)[C@@H]3CC3(F)F)n2)cn1]

or a pharmaceutically acceptable salt thereof, wherein the compound or salt thereof is administered in a dosage of about 10 mg to about 60 mg. In some embodiments, the pharmaceutically acceptable salt is p-toluenesulfonic acid salt.

Embodiments of the present disclosure also include the administration of an effective amount of Brepocitinib to the subject which has one or more effects selected from the group consisting of: decreases or inhibits IFN-I activity in the subject; decreases or inhibits cytokine signaling dysregulation implicated in DM; prevents or reduces damage in human myocytes and microvasculature; and interferes with DM life cycle. In some embodiments, the compound is administered orally.

In some embodiments, the compound is administered in a dosage of about 30 mg. In some embodiments, the compound is administered in a dosage of less than about 30 mg. In some embodiments, the compound is administered in a dosage of about 15 mg.

In some embodiments, the compound administered is taken daily. In some embodiments, the compound administered is in divided doses administered two, three or four times per day.

In some embodiments, the compound is administered for less than or about 104 weeks. In some embodiments, the compound is administered for more than or about 4 weeks.

In some embodiments, the subject is a mammal.

In some embodiments, the mammal is a human.

In some embodiments, the mammal is a canine.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1: A schematic representation of the testing methodology for the use of Brepocitinib in preventing IFN-1 induced damage in cultured myocytes and endothelial cells.

FIG. 2: Illustrates different JAK paring combinations used by various cytokine receptors. Upon binding of the cytokine to its receptor, the associated JAKs are activated and phosphorylate each other and the receptor at specific tyrosine sites. The phosphorylated receptors serve as docking sites for the STAT family (STAT1, STAT2, STAT3, STAT4, STAT5a, STAT5b, and STAT6) of transcription factors. The STATs are then phosphorylated by the co-localized JAKs, which stabilize homo- or hetero-dimeric complexes that translocate to the nucleus where they bind to specific binding sites and modulate transcription of a range of target genes.

FIG. 3: Demonstration of Brepocitinib efficacy in overlapping pathogenic cytokine profiles in various study populations.

FIG. 4: Brepocitinib inhibited cytokine signaling pathways implicated in DM that engaged TYK2 and/or JAK1, including IFNα, IFNg, IL-12, and IL-23. Estimated inhibition of these cytokine signaling pathways at the concentration average (CAVG) of Brepocitinib 30 mg QD.

FIG. 5: Demonstration of the dose dependent efficacy of Brepocitinib.

FIG. 6: Illustrates Brepocitinib ability to rapid dissolve from immediate-release (IR) tablets (>85% in 30 minutes) at 30 mg tablet in pH 6.8 phosphate buffer using USP Apparatus II, 75 rpm.

FIG. 7: Shows representative images of Human skeletal muscle myoblasts (HSMM) immunofluorescence staining that were cultured and differentiated into myotubes and treated with DMSO (vehicle control) or IFN-I (recombinant Human IFN-α A and Human IFN-α D) to induce cellular damage (left image) and a bar graph indicating the effects of Brepocitinib on IFN-I induced myotube damage (right image).

FIG. 8: Shows representative images of Human dermal microvascular endothelial cells (HMEC-1) that were cultured and treated showing the vascular network establishment (left image) and a bar graph indicating the effects of Brepocitinib on IFN-I induced endothelial cell damage (right image).

FIG. 9: Shows a bar graph comparing percentage inhibition values of Brepocitinib 30 mg QD, Brepocitinib 15 mg QD, and Tofacitinib 5 mg BID, respectively, in various DM-relevant cytokines based on cross-trial comparisons.

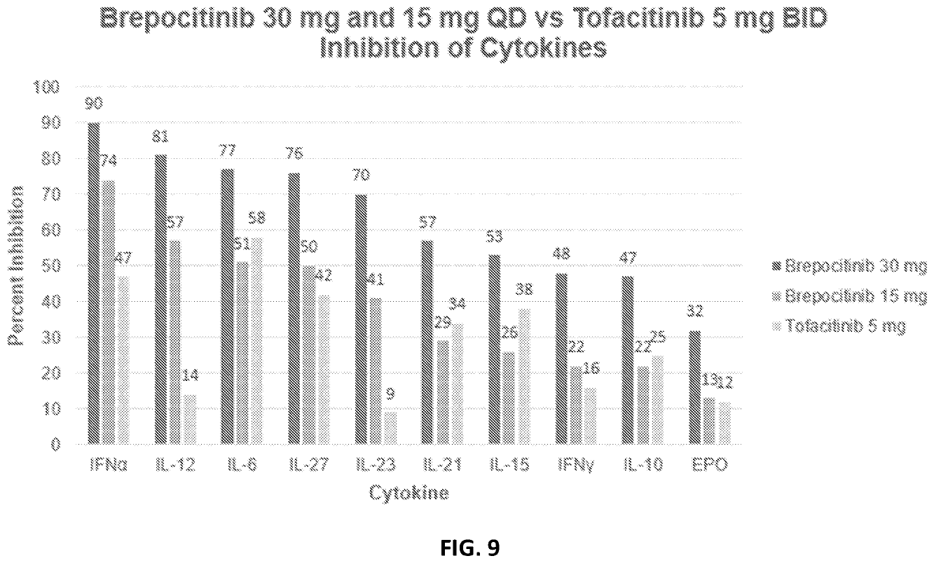

FIG. 10: Shows a bar graph comparing Brepocitinib 30 mg and Tofacitinib 5 or 10 mg BID in treating patients with psoriatic arthritis, plaque psoriasis, or ulcerative colitis based on cross-trial comparisons.

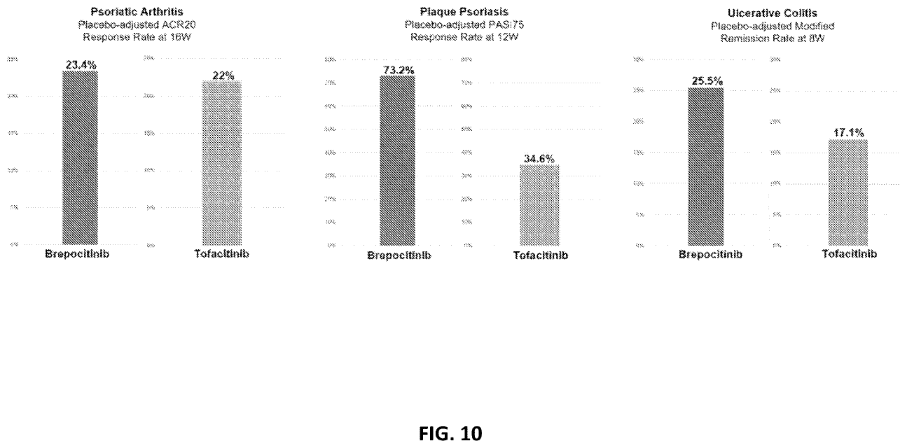

### DETAILED DESCRIPTION

The present disclosure provides methods related to treatment of a skin disease. In particular, the present disclosure provides methods for the oral administration of a JAK inhibitor, or a pharmaceutically acceptable salt thereof for the treatment of dermatomyositis.

Dermatomyositis (DM), an idiopathic inflammatory myopathy, is a chronic and often debilitating condition characterized by a hallmark skin rash (e.g., Gottron's sign, Gottron's papules and heliotrope rash) with perifascicular atrophy and subsequent muscle weakness. Muscle weakness and pain are the most common sources of morbidity, impairing even basic activities of daily life such as dressing, bathing, and walking up and down stairs. Notably, a cutaneous disease, like DM, also results in substantial morbidity and has been shown to dramatically impact patients' quality of life, even more so than other severe skin diseases. Beyond skin and muscle, other systemic manifestations include interstitial lung disease (ILD), arthralgia or arthritis, dysphagia, cardiovascular disease (myocarditis, heart failure, arrythmias, etc.), and an increased risk of malignancy, which further contribute to disease-related morbidity and mortality. Additionally, some patients with DM may become disabled by irreversible muscle damage and/or disfigured by cutaneous disease manifestations.

The pathogenesis of DM involves dysregulation of type 1 interferon (IFN-1) signaling and multiple other cytokines include but, are not limited to, IL-12, IL-23, IL-6, IL-4, IL-22, IFN-γ, IL-15 and IL-21. The kinases, TYK2 and JAK1, are essential signaling pathway of these cytokines. Myositis-specific and myositis-associated antibodies have also been characterized in patients with DM, including anti-TIF-1, the most common antibody in DM patients. Notably, these antibodies often correlate to specific phenotypes, allowing clinicians to better prognosticate and manage patients with DM. For example, some DM patients have high expression levels of anti-melanoma differentiation-associated gene 5 (MDA5) autoantibodies and are particularly prone to rapidly progressive interstitial lung disease (RP-ILD). Calcinosis cutis, which is often associated with the presence of anti-NXP2 antibodies, is another serious manifestation of DM that can occur in up to 20% of adult DM patients.

Brepocitinib, an orally administered small molecule inhibitor of TYK2 and JAK1, is in development for the treatment of DM. It is expected to reduce a range of pro-inflammatory cytokine signaling, including type I & II IFN, IL-6, and IL-12/IL-23 and ameliorate the symptoms of the disease. This formulation is suitable for once-daily dosing. The JAK family contains four non-receptor tyrosine kinases of similar structure and function: JAK1, JAK2, JAK3, and TYK2. Each janus kinase (JAK) family member has a characteristic association with the intracellular domain of one of almost 40 cytokine receptor monomers. The signaling of cytokines is mediated through this JAK-cytokine receptor complex, thereby enabling the mechanistic feasibility for inhibition of multiple cytokines with a single molecule, that is a JAK inhibitor.

These findings on Brepocitinib are key as previous findings have shown that one of the most debilitating aspects of dermatomyositis DM is muscle weakness, which results from perifascicular atrophy and significantly impacts the quality of life for affected individuals. The data has demonstrated Brepocitinib ability to prevent IFN-I-induced damage in both myocytes and microvasculature in culture at clinically relevant concentrations. This supports the pharmacological rationale for considering Brepocitinib as a potential treatment for DM.

As disclosed further herein, the various embodiments of the present disclosure include method for treating DM comprising administering to a subject in need of treatment thereof an effective amount Brepocitinib or a pharmaceutically acceptable salt thereof, wherein the compound or salt thereof is administered in a dosage of about 10 mg to about 60 mg in a manner that is significantly more efficient than currently available oral administration treatments for DM.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in some embodiments” as used herein does not necessarily refer to the same embodiment, though it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “dermatomyositis” refers to a rare autoimmune inflammatory disease characterized by skin rashes, perifascicular atrophy of muscle fibers, and subsequent muscle weakness. Muscle weakness typically affects the proximal muscles closest to the trunk, such as those in the hips, thighs, shoulders, upper arms, and neck. This weakness is usually symmetrical, meaning it affects both sides of the body. The skin rash can present as a heliotrope rash, which is a violet or dusky red rash that appears on the upper eyelids, sometimes with swelling. Gottron's papules are red or violet, scaly, raised bumps found over the knuckles, elbows, knees, and other joints. The rash can also appear on the upper chest and back (V-sign) or shoulders and upper arms (shawl-sign). Systemic symptoms of dermatomyositis include fatigue, joint pain and swelling, dysphagia (difficulty swallowing), and pulmonary issues.

Diagnosis of dermatomyositis includes blood tests to check for elevated muscle enzymes (e.g., creatine kinase) and autoantibodies, electromyography (EMG) to measure electrical activity in muscles, muscle biopsy to examine tissue for signs of inflammation and damage, and imaging, such as MRI, to detect muscle inflammation. Embodiments of the present disclosure include a method of administering Brepocitinib for treating dermatomyositis.

As used herein, the terms “providing,” “administering,” and “introducing,” are used interchangeably herein and refer to the placement of the proteins or systems of the disclosure into a subject by a method or route which results in at least partial localization to a desired site. Administration can use any appropriate route which results in delivery to a desired location in the subject.

As used herein, the term “life cycle” is used to refer to the stages and progression of a particular disease from its initial introduction or onset through its development, transmission, effects on the host or population, and, in some cases, resolution. The life cycle of a disease encompasses the complete course of a disease, including its origin, spread, impact, and potential outcomes. The life cycle phases of a disease would include: the disease onset, transmission of the disease, the incubation period, the symptomatic phase, the recovery or chronic phase, the spread or outbreak of the disease, the immunity or resistance period of the disease, the immunity or resistance period of the disease, the decline and elimination of the disease as well as the potential resurgence of the disease.

As used herein, the phrase “pharmaceutically acceptable carrier” is used to refer to a material that is compatible with a recipient subject, such as a mammal, more particularly a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The terms “carrier”, “adjuvant”, or “vehicle” are used interchangeably herein, and include any and all solvents, diluents, and other liquid vehicles, dispersion or suspension aids, surface active agents, isotonic agents, thickening or emulsifying agents, preservatives, solid binders, lubricants and the like, as suited to the particular dosage form desired. Remington: The Science and Practice of Pharmacy. 20th Ed., ed. A. Gennaro, Lippincott Williams & Wilkins, 2000 discloses various carriers used in formulating pharmaceutically acceptable compositions and known techniques for the preparation thereof. Except insofar as any conventional carrier medium is incompatible with the compounds of the present disclosure, such as by producing any undesirable biological effect or otherwise interacting in a deleterious manner with any other component(s) of the pharmaceutically acceptable composition, its use is contemplated to be within the scope of this disclosure. Some examples of materials which can serve as pharmaceutically acceptable carriers include, but are not limited to, ion exchangers, alumina, aluminum stearate, lecithin, serum proteins, such as human serum albumin, buffer substances such as disodium hydrogen phosphate, potassium hydrogen phosphate, sodium carbonate, sodium bicarbonate, potassium carbonate, potassium bicarbonate, magnesium hydroxide and aluminum hydroxide, glycine, sorbic acid, or potassium sorbate, partial glyceride mixtures of saturated vegetable fatty acids, water, pyrogen-free water, salts or electrolytes such as protamine sulfate, disodium hydrogen phosphate, potassium hydrogen phosphate, sodium chloride, and zinc salts, colloidal silica, magnesium trisilicate, polyvinyl pyrrolidone, polyacrylates, waxes, polyethylene-polyoxypropylene-block polymers, wool fat, sugars such as lactose, glucose, sucrose, starches such as corn starch and potato starch, cellulose and its derivatives such as sodium carboxymethyl cellulose, ethyl cellulose and cellulose acetate, powdered tragacanth; malt, gelatin, talc, excipients such as cocoa butter and suppository waxes, oils such as peanut oil, cottonseed oil, safflower oil, sesame oil, olive oil, corn oil and soybean oil, glycols such as propylene glycol and polyethylene glycol, esters such as ethyl oleate and ethyl laurate, agar, alginic acid, isotonic saline, Ringer's solution, alcohols such as ethanol, isopropyl alcohol, hexadecyl alcohol, and glycerol, cyclodextrins, lubricants such as sodium lauryl sulfate and magnesium stearate, petroleum hydrocarbons such as mineral oil and petrolatum. Coloring agents, releasing agents, coating agents, sweetening, flavoring and perfuming agents, preservatives and antioxidants can also be present in the composition, according to the judgment of the formulator.

As used herein, the phrase, “pharmaceutically acceptable salt” refers to those salts which arc, within the scope of sound medical judgment, suitable for use in contact with the tissues of subjects (e.g., humans and other mammals) without undue toxicity, irritation, allergic response and the like, and are commensurate with a reasonable benefit/risk ratio. In some embodiments, a pharmaceutically acceptable salt of Brepocitinib is derived from an inorganic or organic acid or base. For reviews of suitable salts, see, e.g., Berge et al, J. Pharm. Sci. 66:1-19 (1977) and Remington: The Science and Practice of Pharmacy. 20th Ed., ed. A. Gennaro, Lippincott Williams & Wilkins, 2000. Examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, mesylate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Suitable base addition salts include, without limitation, ammonium salts, alkali metal salts, such as sodium and potassium salts, alkaline earth metal salts, such as calcium and magnesium salts, salts with organic bases, such as dicyclohexylamine, N-methyl-D-glucamine, t-butylamine, ethylene diamine, ethanolamine, and choline, and salts with amino acids such as arginine, lysine, and so forth.

Also, basic nitrogen-containing groups may be quaternized with such agents as lower alkyl halides, such as methyl, ethyl, propyl, and butyl chlorides, bromides, and iodides; dialkyl sulfates, such as dimethyl, diethyl, dibutyl and diamyl sulfates, long chain halides such as decyl, lauryl, myristyl and stearyl chlorides, bromides, and iodides, aralkyl halides, such as benzyl and phenethyl bromides and others. Water or oil-soluble or dispersible products are thereby obtained.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus or rhesus monkey, chimpanzee, etc.) and a human). In some embodiments, the subject may be a human or a non-human.

The phrase “therapeutically effective” and “effective amount” refer to a benefit including, but not limited to, the treatment or amelioration of symptoms of Dermatomyositis as discussed herein. It will be appreciated that the therapeutically effective amount or the amount of one or more agents required to provide a therapeutic effect will vary depending upon the intended application (in vitro or in vivo), or the subject and disease condition being treated (e.g., nature of the severity of the condition to be treated, the particular inhibitor, the route of administration and the age, weight, general health, and response of the individual subject), which can be readily determined by a person of skill in the art. For example, an amount of Brepocitinib is therapeutically effective if it is sufficient to effect the treatment or amelioration of symptoms of dermatomyositis as discussed herein.

As used herein, whether by themselves or in conjunction with another term or terms, “treats,” “treating,” “treated,” and “treatment,” refer to and include ameliorative, palliative, and/or curative uses and results, or any combination thereof. In other embodiments, the methods described herein can be used prophylactically, that is, preventatively. It should be understood that “prophylaxis” or a prophylactic use or result do not refer to nor require absolute or total prevention (i.e., a 100% preventative or protective use or result). As used herein, prophylaxis or a prophylactic (preventative) use or result refers to uses and results in which administration of a compound, therapeutic agent or composition diminishes or reduces the severity of a particular condition, symptom, disorder, or disease described herein; diminishes or reduces the likelihood of experiencing a particular condition, symptom, disorder, or disease described herein; or delays the onset or relapse (reoccurrence) of a particular condition, symptom, disorder, or disease described herein; or any combination of the foregoing.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Oral Administration of Brepocitinib

Embodiments of the present disclosure include a method for comprising administering to a subject in need of treatment thereof (e.g., such as a subject suffering from dermatomyositis) an effective amount of [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl] methanone (Brepocitinib)

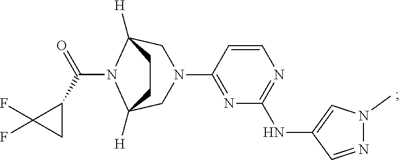 [SMILES: Cn1cc(Nc2nccc(N3C[C@H]4CC[C@@H](C3)N4C(=O)[C@@H]3CC3(F)F)n2)cn1]

or a pharmaceutically acceptable salt thereof. The Brepocitinib (referred to interchangeably herein as “the compound”) or a pharmaceutically acceptable salt thereof can be administered to a subject in a dose of about 10 mg to about 60 mg. For example, the compound or pharmaceutically acceptable salt thereof is administered in a dose of about 10 mg to about 50 mg. In some embodiments, the compound or pharmaceutically acceptable salt thereof is administered in a dose of about 10 mg to about 40 mg. In some embodiments, the compound or pharmaceutically acceptable salt thereof is administered in a dose of about 10 mg to about 30 mg. In some embodiments, the compound or pharmaceutically acceptable salt thereof is administered in a dosage of about 10 mg to about 20 mg. In some embodiments, the compound or pharmaceutically acceptable salt thereof is administered in a dose of about 15 mg to about 60 mg. In some embodiments, the compound or pharmaceutically acceptable salt thereof is administered in a dosage of about 15 to about 50 mg. In some embodiments, the compound or pharmaceutically acceptable salt thereof is administered in a dose of about 15 to about 40 mg. In some embodiments, the compound or pharmaceutically acceptable salt thereof is administered in a dosage of about 15 to about 30 mg. In some embodiments, the compound or pharmaceutically acceptable salt thereof is administered in a dose of about 15 to about 20 mg. In some embodiments, the compound or pharmaceutically acceptable salt thereof is administered in a dose of about 20 mg to about 60 mg. In some embodiments, the compound or pharmaceutically acceptable salt thereof is administered in a dose of about 30 mg to about 60 mg. In some embodiments, the compound or pharmaceutically acceptable salt thereof is administered in a dose of about 40 mg to about 60 mg. In some embodiments, the compound or pharmaceutically acceptable salt thereof is administered in a dose of about 50 mg to about 60 mg. In some embodiments, the compound or pharmaceutically acceptable salt thereof is administered in a dose of about 20 mg to about 50 mg. In some embodiments, the compound or pharmaceutically acceptable salt thereof is administered in a dose of about 30 mg to about 40 mg. In some embodiments, the pharmaceutically acceptable pharmaceutically acceptable salt is p-toluenesulfonic acid pharmaceutically acceptable salt. In some embodiments, p-toluenesulfonic acid pharmaceutically acceptable salt is (1S)-2,2-difluorocyclopropyl][3-[2-[(1-methyl-1H pyrazol-4-yl)amino]-4-pyrimidinyl]-3,8-diazabicyclo[3.2.1]oct-8-yl]-methanone,4-methylbenzenesulfonate (1:1) with the structure:

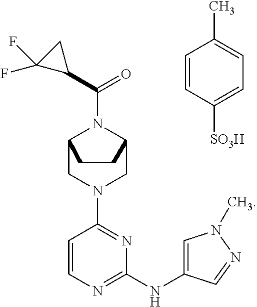

Methods for making Brepocitinib are described at least in WO 2016/027195 and U.S. Pat. No. 9,663,526, the contents of which are herein incorporated by reference.

Embodiments of the present disclosure also include the administration of an effective amount of Brepocitinib to the subject has one or more effects selected from the group consisting of: decreases or inhibits IFN-I activity in the subject; decreases or inhibits cytokine signaling dysregulation implicated in dermatomyositis (DM); prevents or reduces damage in human myocytes and microvasculature; and interferes with DM life cycle. In some embodiments, the compound is administered orally.

In some embodiments, the compound or pharmaceutically acceptable salt thereof is administered in a dosage that is about 60 mg. In some embodiments, the compound or pharmaceutically acceptable salt thereof is administered in a dosage that is about 50 mg. In some embodiments, the compound or pharmaceutically acceptable salt thereof is administered in a dosage that is about 40 mg. In yet other embodiments, embodiments, the compound, or pharmaceutically acceptable salt thereof is administered in a dosage that is about 30 mg. In some embodiments, the compound or pharmaceutically acceptable salt thereof is administered in a dosage that is about 25 mg. In some embodiments, the compound or pharmaceutically acceptable salt thereof is administered in a dosage that is about 20 mg. In some embodiments, the compound or pharmaceutically acceptable salt thereof is administered in a dosage that is about 15 mg. In some embodiments, the compound or pharmaceutically acceptable salt thereof is administered in a dosage that is about 10 mg. In some embodiments, the compound or pharmaceutically acceptable salt thereof is administered in a dosage that is about 5 mg.

In some embodiments, the compound or pharmaceutically acceptable salt thereof is administered in a dosage that is less than about 60 mg. In some embodiments, the compound or pharmaceutically acceptable salt thereof is administered in a dosage that is less than about 50 mg. In some embodiments, the compound or pharmaceutically acceptable salt thereof is administered in a dosage that is less than about 40 mg. In yet other embodiments, embodiments, the compound or pharmaceutically acceptable salt thereof is administered in a dosage that is less than about 30 mg. In some embodiments, the compound or pharmaceutically acceptable salt thereof is administered in a dosage that is less than about 25 mg. In some embodiments, the compound or pharmaceutically acceptable salt thereof is administered in a dosage that is less than about 20 mg. In some embodiments, the compound or pharmaceutically acceptable salt thereof is administered in a dosage that is less than about 15 mg. In some embodiments, the compound or pharmaceutically acceptable salt thereof is administered in a dosage that is less than about 10 mg. In some embodiments, the compound or pharmaceutically acceptable salt thereof is administered in a dosage that is less than about 5 mg.

In some embodiments, the compound or pharmaceutically acceptable salt thereof administered is taken daily. In some embodiments, the compound or pharmaceutically acceptable salt thereof is taken once daily in the dosages recited above. In some embodiments, the compound or pharmaceutically acceptable salt thereof administered is in divided doses administered two, three or four times per day in the dosages recited above.

In some embodiments, the compound or pharmaceutically acceptable salt thereof is administered for less than or about 104 weeks. For example, the compound or pharmaceutically acceptable salt thereof is administered for less than or about 100 weeks. In some embodiments, the compound or pharmaceutically acceptable salt thereof is administered for less than or about 90 weeks. In some embodiments, the compound or pharmaceutically acceptable salt thereof is administered for less than or about 80 weeks. In some embodiments, the compound or pharmaceutically acceptable salt thereof is administered for less than or about 70 weeks. In some embodiments, the compound or pharmaceutically acceptable salt thereof is administered for less than or about 60 weeks. In some embodiments, the compound or pharmaceutically acceptable salt thereof is administered for less than or about 50 weeks. In some embodiments, the compound or pharmaceutically acceptable salt thereof is administered for less than or about 40 weeks. In some embodiments, the compound or pharmaceutically acceptable salt thereof is administered for less than or about 30 weeks. In some embodiments, the compound or pharmaceutically acceptable salt thereof is administered for less than or about 20 weeks. In some embodiments, the compound or pharmaceutically acceptable salt thereof is administered for less than or about 10 weeks. In some embodiments, the compound or pharmaceutically acceptable salt thereof is administered for less than or about 5 weeks. In some embodiments, the compound or pharmaceutically acceptable salt thereof is administered for less than or about 4 weeks. In some embodiments, the compound is administered for less than or about 3 weeks. In some embodiments, the compound or pharmaceutically acceptable salt thereof is administered for less than or about 2 weeks. In some embodiments, the compound or pharmaceutically acceptable salt thereof is administered for less than or about 1 week.

In some embodiments, the compound or pharmaceutically acceptable salt thereof is administered for more than or about 4 weeks. For example, the compound or pharmaceutically acceptable salt thereof is administered for more than or about 5 weeks. In some embodiments, the compound or pharmaceutically acceptable salt thereof is administered for more than or about 6 weeks. In some embodiments, the compound or pharmaceutically acceptable salt thereof is administered for more than or about 7 weeks. In some embodiments, the compound or pharmaceutically acceptable salt thereof is administered for more than or about 8 weeks. In some embodiments, the compound or pharmaceutically acceptable salt thereof is administered for more than or about 9 weeks. In some embodiments, the compound or pharmaceutically acceptable salt thereof is administered for more than or about 10 weeks. In some embodiments, the compound or pharmaceutically acceptable salt thereof is administered for more than or about 20 weeks. In some embodiments, the compound or pharmaceutically acceptable salt thereof is administered for more than or about 30 weeks. In some embodiments, the compound or pharmaceutically acceptable salt thereof is administered for more than or about 40 weeks. In some embodiments, the compound or pharmaceutically acceptable salt thereof is administered for more than or about 50 weeks. In some embodiments, the compound or pharmaceutically acceptable salt thereof is administered for more than or about 60 weeks. In some embodiments, the compound or pharmaceutically acceptable salt thereof is administered for more than or about 70 weeks. In some embodiments, the compound or pharmaceutically acceptable salt thereof is administered for more than or about 80 weeks. In some embodiments, the compound or pharmaceutically acceptable salt thereof is administered for more than or about 90 weeks. In some embodiments, the compound or pharmaceutically acceptable salt thereof is administered for more than or about 100 weeks. In some embodiments, the compound or pharmaceutically acceptable salt thereof is administered for more than or about 101 weeks. In some embodiments, the compound or pharmaceutically acceptable salt thereof is administered for more than or about 102 weeks. In some embodiments, the compound or pharmaceutically acceptable salt thereof is administered for more than or about 103 weeks. In some embodiments, the compound or pharmaceutically acceptable salt thereof is administered for more than or about 104 weeks.

In some embodiments, the compound or pharmaceutically acceptable salt thereof is administered for about 50 weeks, about 51 weeks, about 52 weeks, about 53 weeks, about 54 weeks, about 55 weeks, about 56 weeks, about 57 weeks, about 58 weeks, about 59 weeks, or about 60 weeks.

The Brepocitinib or pharmaceutically acceptable salt can be administered by any methods known to one skilled in the art. For example, Brepocitinib or pharmaceutically acceptable salt thereof can be administered in the form of a composition, such as, for example, a pharmaceutical composition of Brepocitinib or pharmaceutically acceptable salt and a pharmaceutically acceptable carrier, such as those described herein. In some embodiments, the pharmaceutical composition is suitable for oral administration. In some embodiments, the pharmaceutical composition is a tablet for oral administration, such as an enteric coated tablet. In some other embodiments, the pharmaceutical composition is a liquid dosage form for oral administration. In some embodiments, these composition optionally further comprises one or more additional therapeutic agents.

The pharmaceutical compositions described herein can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired.

In some embodiments, pharmaceutical compositions are formulated for pharmaceutical administration to a mammal, such as a human or a canine. Such pharmaceutical compositions may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intravenously, or subcutaneously. The formulations of the present disclosure may be designed to be short-acting, fast-releasing, or long-acting. Still further, compounds can be administered in a local rather than systemic means, such as administration (e.g., by injection) at a tumor site.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push or may be administered by continuous infusion.

In order to prolong the effect of a compound or therapeutic agent (e.g., Brepocitinib or pharmaceutically acceptable salt thereof), it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the osimertinib and/or alisertib with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the therapeutic agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The Brepocitinib or pharmaceutically acceptable salt can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the therapeutic agents described herein include ointments, pastes, creams, lotions, gels, powders, solutions, sprays, inhalants, or patches. The active component is admixed under sterile conditions with a pharmaceutically acceptable carrier and any needed preservatives or buffers as may be required. Ophthalmic formulation, car drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compositions for use in the method of the present disclosure may be formulated in unit dosage form for ease of administration and uniformity of dosage. The phrase “unit dosage form” as used herein refers to a physically discrete unit of agent appropriate for the subject to be treated. It will be understood, however, that the total daily usage of the compounds and pharmaceutical compositions described herein will be decided by the attending physician within the scope of sound medical judgment. A unit dosage form for parenteral administration may be in ampoules or in multi-dose containers.

### Example 1

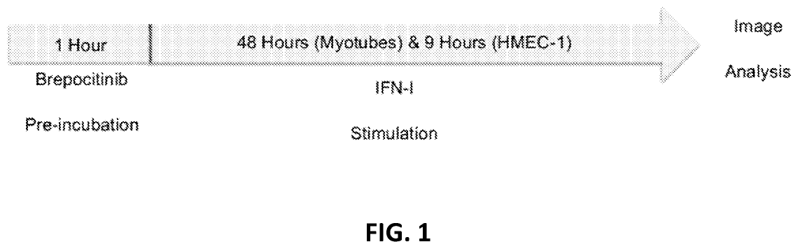

Timeline for Brepocitinib efficacy evaluation for IFN-I induced pathological changes characteristic of DM in vitro. As shown in the schematic image of FIG. 1, human skeletal muscle myoblasts (HSMM) were cultured and differentiated into myotubes then treated with vehicle control or IFN-I (recombinant Human IFN alpha A and Human IFN alpha D) to induce cellular damage. Myotubes were also preincubated for 1 hour with Brepocitinib (1 μM or 130 nM, the latter is equivalent to the average free plasma concentration after administration of 30 mg once-daily) prior to IFN-I treatment. Immunofluorescence staining followed by image analysis to determine myosin 4 surface area were conducted after 48 hours of IFN-I treatment. Human dermal microvascular endothelial cells (HMEC-1) were cultured and, once vascular networks were established, cells were treated with IFN-I or vehicle control. Cells were also preincubated for 1 hour with Brepocitinib. Under light microscopy, 9 hours after treatment, the number of nodes, master segment length, and total mesh area were analyzed.

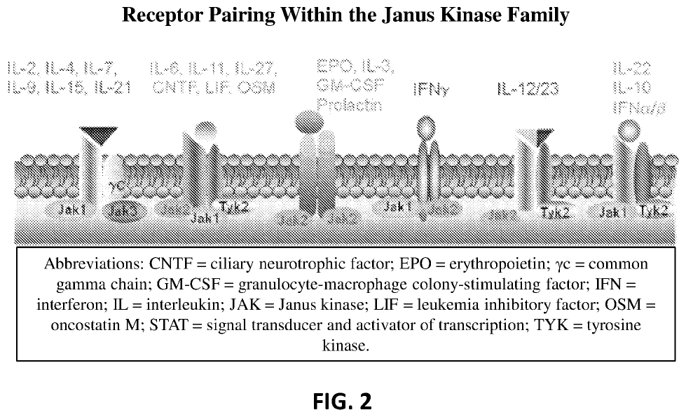

Primary Pharmacodynamics. The JAK family, including JAK1, JAK2, JAK3, and JYK2 is a group of cytoplasmic tyrosine kinases that mediate signal transduction of cytokine receptors. FIG. 2 illustrates different JAK paring combinations used by various cytokine receptors. Upon binding of the cytokine to its receptor, the associated JAKs are activated and phosphorylate each other and the receptor at specific tyrosine sites. The phosphorylated receptors serve as docking sites for the STAT family (STAT1, STAT2, STAT3, STAT4, STAT5a, STAT5b, and STAT6) of transcription factors. The STATs are then phosphorylated by the co-localized JAKs, which stabilize homo- or hetero-dimeric complexes that translocate to the nucleus where they bind to specific binding sites and modulate transcription of a range of target genes.

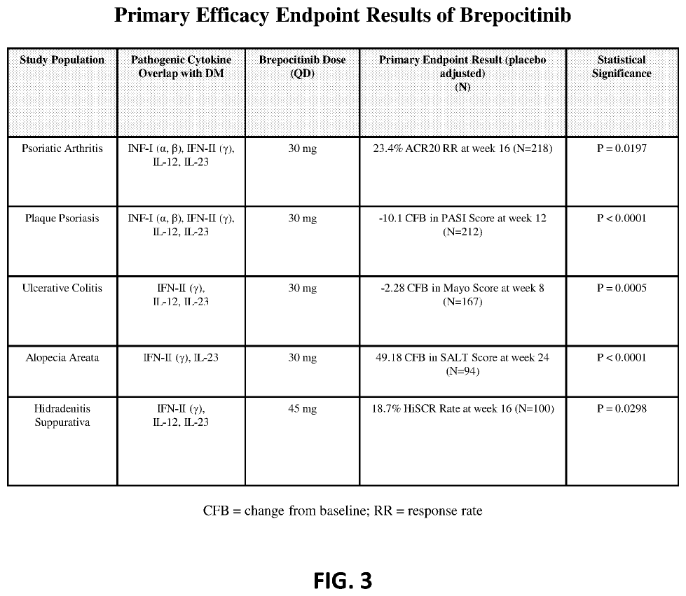

Primary efficacy endpoints with Brepocitinib. As shown in FIG. 3, Brepocitinib has been investigated in 5 completed Phase 2 placebo-controlled studies in psoriatic arthritis, plaque psoriasis, ulcerative colitis, alopecia areata, and hidradenitis suppurativa. In each, clinically meaningful and statistically significant results were achieved across a diverse array of organ systems (e.g., skin, joints, gastrointestinal). These clinical data, along with the overlapping pathogenic cytokine profile, support the rationale for evaluating Brepocitinib in DM.

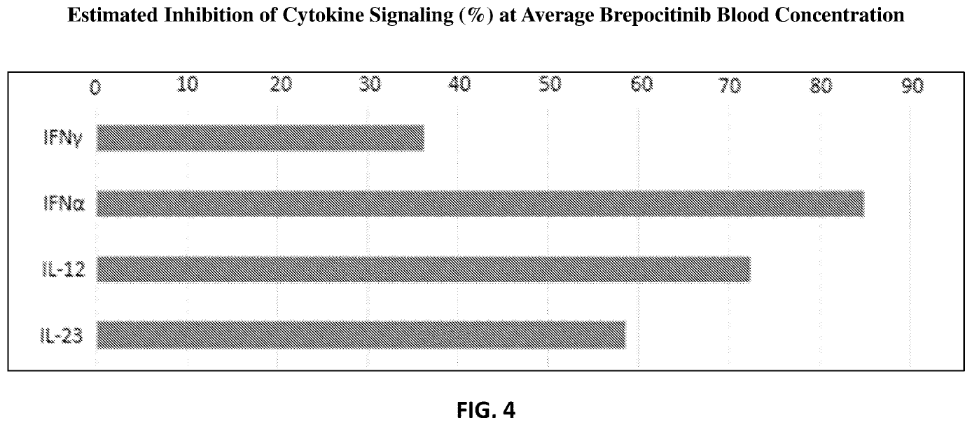

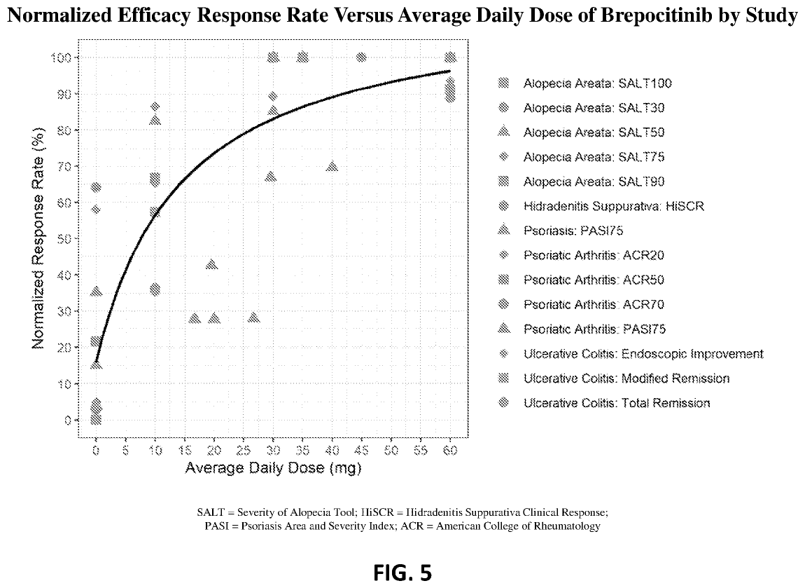

Pharmacologic and clinical rationale of Brepocitinib. In vitro, Brepocitinib potently and selectively inhibits TYK2 and JAK1, with IC₅₀ values (23 and 17 nM, respectively) at least 3-fold lower than those for JAK2 and JAK3. In human whole blood, Brepocitinib inhibited cytokine signaling pathways implicated in DM that engage TYK2 and/or JAK1, including IFNα, IFNg, IL-12, and IL-23. Estimated inhibition of these cytokine signaling pathways at the Cavg of Brepocitinib 30 mg once daily (QD) is shown in FIG. 4. Across Phase 2 studies, Brepocitinib demonstrated dose-dependent efficacy. The relationship between normalized responder rate and average daily Brepocitinib dose is shown in FIG. 5. The dose resulting in 50% maximal response was determined to be 15 mg QD with generally minimal increased efficacy observed at doses greater than 30 mg QD.

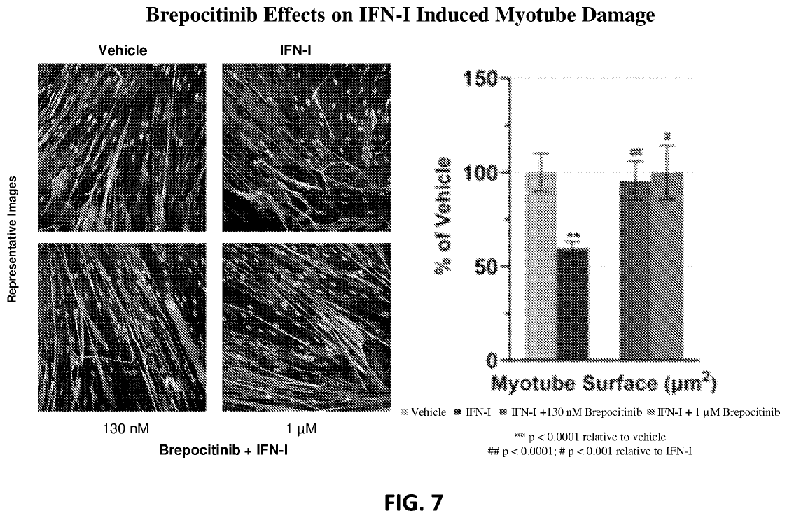

Brepocitinib prevents IFN-1 damage in cultured cells. As shown in FIG. 7 myosin surface area was reduced by ~40%, relative to vehicle treated control, in myotubes exposed to IFN-I (p<0.0001). This cytokine induced damage was almost completely prevented by Brepocitinib preincubation (both 130 nM and 1 μM) with mean myosin surface areas of 94.1% and 100% of the vehicle control, respectively. The differences between IFN-I treatment alone and Brepocitinib were significant (p<0.0001 [1 μM] and P<0.001 [130 nM]).

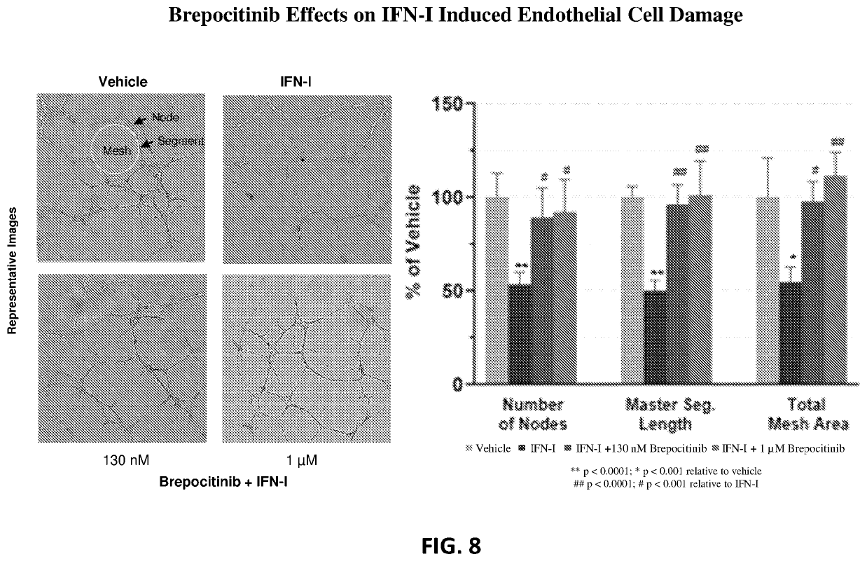

Similarly, as shown in FIG. 8, HMEC-1 exposure to IFN-I significantly reduced the mean number of nodes, mean master segment length, and mean total mesh area by 47 to 50% relative to vehicle control (p<0.0001 nodes and segments, p<0.001 mesh area). With Brepocitinib preincubation, this damage was prevented with the mean number of nodes, master segment lengths, and total mesh area ranging from 89 to 111% of vehicle control. These differences were statistically different from the IFN-I treatment at both Brepocitinib concentrations and all endpoints.

### Example 2

Phase 3, Randomized, Double-Blind, Placebo-Controlled Study to Investigate the Efficacy and Safety of Oral Brepocitinib in Adults with Dermatomyositis

Overall Design This is a prospective, parallel-group, randomized, double-blind, placebo-controlled, international, multicenter Phase 3 study with a 52-week double-blind treatment period (Blinded Treatment Period) followed by a 52-week open-label treatment period (OLE Period). The study population comprises individuals with definite or probable idiopathic inflammatory myopathy (IIM) who meet the subclassification criteria for classic dermatomyositis. All participants may continue to receive permitted dermatomyositis standard of care medications.

### Study Details Include:

The study duration will be up to 116 weeks and consists of the following segments: up to 8 weeks for screening (Screening Period), then 52 weeks of double-blind treatment with placebo or active study drug (Blinded Treatment Period), then 52 weeks of active study drug (OLE Period), then 4-weeks for safety follow-up (Off-Drug Follow-Up Period).
For participants using corticosteroids at baseline, corticosteroid tapering (between Weeks 12 and 36) to an oral corticosteroid dose of prednisone≤5 mg/day (or equivalent) is required in all participants, except for participants receiving rescue therapy. Tapering below prednisone 5 mg/day (or equivalent) is at the discretion of the investigator.
The Off-Drug Follow-Up Period will either immediately follow the Blinded Treatment Period (if the participant does not continue into the OLE Period) or the OLE Period.
The duration of treatment with study drug is up to 104 weeks (an initial 52 weeks with double-blind study drug [active or placebo] plus an additional 52 weeks of open-label study drug [active]).
The visit frequency is every 4-6 weeks during the Blinded Treatment Period and every 4-12 weeks during the OLE Period.

Number of Participants. Approximately 225 participants will be randomized (enrolled) at approximately 110 sites globally.

Randomization to Study Treatment. Participants who are determined to be eligible for the study during the Screening Period will be randomized 1:1:1 at baseline (Day 1/Visit 2) to one of the following intervention groups (for study drug administered during the Double Blind Treatment Period):
Arm 1: Brepocitinib 30 mg PO QD for 52 weeks (n=75);
Arm 2: Brepocitinib 15 mg PO QD for 52 weeks (n=75);
Arm 3: placebo PO QD for 52 weeks (n=75).

During the OLE Period, all participants (regardless of original treatment assignment) will receive Brepocitinib 30 mg PO QD for up to 52 weeks.

Throughout the study, all treatment arms may receive ongoing standard of care oral background therapy for dermatomyositis.

Randomization will be stratified based on the following baseline factor:
PhGA-VAS baseline value (0 to 4.9 cm, 5 to 10 cm).

Study drugs and Concomitant therapy. For the purposes of this protocol, “investigational medicinal product” (IMP) is defined as Brepocitinib. “Study drug” is defined as either IMP (Brepocitinib) or placebo intended to be administered to a study participant according to the study protocol.

Study Drugs Administered. Participants should take the study drug by mouth once daily. For participants with difficulty swallowing the study drug tablets, refer to the Pharmacy Manual for guidance.

Participants will be encouraged to take the study drug in the morning after breakfast whenever possible; however, the study drug may be taken with or without food.

At baseline (Day 1/Visit 2), participants will receive their first dose of double-blinded study drug at the site and a supply of study drug to take home. For Week 4/Visit 3 and Week 24/Visit 7, participants should be instructed to refrain from dosing at home on the day of the visit and to take the study drug at the site during the visit. For Week 52/Visit 12, participants should be instructed to refrain from dosing at home on the day of the visit; participants who do not continue into the OLE Period will have taken their last dose of study drug on the day before Week 52/Visit 12, whereas participants who continue into the OLE Period will receive their first dose of open-label study drug at the site.

If a dose is missed and the interval to the next dose is less than 8 hours, the missed dose should not be administered, and should be recorded as a missed dose.

Brepocitinib and matching placebo are provided as tablets for oral administration. The study drug is packaged to maintain blinding as appropriate and labeled according to regional regulatory requirements. During the Blinded Treatment Period, participants will receive doses of Brepocitinib 15 mg, Brepocitinib 30 mg, or placebo. During the OLE Period, all participants will receive Brepocitinib 30 mg.

As the study progresses, additional Brepocitinib formulations or packaging configurations may be introduced to enhance participant convenience or simplify study drug administration.

Allocation to Study Drug. All participants will be centrally assigned to randomized blinded study drug using an interactive response technology (IRT). The log-in information and directions for the IRT will be provided to each site prior to enrolling participants. The site personnel (study coordinator or specified designee) will be required to enter or select information including but not limited to the user's identification (ID) and password, the protocol number, and the participant number (Subject ID). The site personnel will then be provided with a treatment assignment, randomization number,

Randomization will be stratified based on the following factor:
PhGA-VAS baseline value (0 to 4.9 cm, 5 to 10 cm).

All study drug dispensed will be accounted for, including recording study drug dispensed to participants and return of any unused study drug by the participant.

Study drug may be returned to the site via mail or courier service if a participant cannot come to the center and a return of study drug is required (e.g., the participant withdraws consent to participate in the study).

Blinding and Blind Breaking. During the Blinded Treatment Period, the participants, site personnel, investigators, and sponsor personnel are blinded to study drug. The investigators and participants will remain blinded to the study drug received during the Blinded Treatment Period until the study is complete. After all participants have completed Week 52/Visit 12 and had a retest (if necessary), an interim database lock will be declared after the completion of all procedures related to data cleaning, the medical review of the data, agreement of the final participant disposition, and agreement of the analysis populations upon review of all protocol deviations. The sponsor/sponsor's designee will then be granted access to the unblinded database in order to analyze the Blinded Treatment Period data. The 52-week double-blind treatment period data will be summarized in a clinical study report (CSR).

The PK vendor, as an independent party, will be granted access to the randomization codes during the conduct of the trial for analysis purposes. The codes will be filed securely in a manner that ensures that the blind is properly maintained throughout the Blinded Treatment Period. The results from the PK analysis will not be shared until the Week 52 database lock has occurred.

The IRT will be programmed with blind-breaking instructions. Blinding codes should be broken only in exceptional circumstances when knowledge of the actual treatment code is absolutely essential for further management of the participant. In case of an emergency, the investigator has the sole responsibility for determining if unblinding of a participant's study drug assignment is warranted. Participant safety must always be the first consideration in making such a determination. All efforts should be made to contact the medical monitor and/or sponsor immediately (and prior to unblinding) if the need for emergent unblinding of the participant's treatment assignment is required for their safety, unless this could delay emergency treatment for the participant. If a participant's study drug assignment is unblinded, the sponsor must be notified within 24 hours of this occurrence. The date and reason for the unblinding must be recorded in the source documentation and eCRF.

Screening Period. Based on the timing of the onset of the potential participant's dermatomyositis symptoms, the Screening Period may last up to 8 weeks or up to 4 weeks:
Potential participants with onset of dermatomyositis symptoms <3 years prior to screening or if the timing of onset of symptoms is unknown: For such participants, their study visit for screening will be Visit which may occur up to 8 weeks prior to baseline (Day 1/Visit 2). The primary purpose of Visit 1a is to identify individuals at elevated risk for malignancy or rapidly progressive ILD, and assessments during the Screening Period will include a CT (or PET-CT) scan if necessary to help interrogate this potential risk.
Potential participants with onset of dermatomyositis symptoms ≥3 years prior to screening: For such participants, their study visit for screening will be Visit 1b, which may occur up to 4 weeks prior to baseline (Day 1/Visit 2).

Criteria for Temporarily Delaying Enrollment/Randomization. An extension of the Screening Period may be granted after consultation between the investigator and sponsor. Reasons for extending the Screening Period include, but are not limited to, the following:
Results of laboratory tests or other assessments required to determine eligibility are pending (e.g., if a CT [or PET-CT] scan is required and has been performed, but results have not been reported within the designated Screening Period).
Results of eligibility committee review are pending (e.g., if results have been submitted but the committee has not reviewed and made a recommendation within the designated Screening Period).
Clinic visit scheduling is delayed or shipment of supplies to the site (e.g., study drug or laboratory kits) is delayed due to a global crisis or other unforeseeable reasons.

Blinded Treatment Period. The Blinded Treatment Period begins at baseline (Day 1/Visit 2) and ends at Week 52/Visit 12, which is the primary analysis timepoint. After final confirmation of study eligibility participants will be randomized to their study intervention group on Day 1. Once randomized, participants will take their double-blinded study drug, beginning on Day 1, once daily for 52 weeks (with the last dose taken on the day before the visit for Week 52/Visit 12).

During the Blinded Treatment Period, a participant may receive oral steroid-based rescue therapy in addition to study drug.

Inclusion Criteria. An individual will be eligible for participation in this study only if all of the following inclusion criteria are met:

### Age and Sex.

1. Male or female, and the participant must be ≥18 to ≤75 years of age at the time of signing the informed consent form (ICF).

### Type of Participant and Disease Characteristics.

2. Participants with a diagnosis of dermatomyositis according to 2017 EULAR/ACR Classification Criteria for Idiopathic Inflammatory Myopathies

Note: Participants with possible IIM are not eligible. For patients with probable IIM, diagnosis confirmation from the Independent Eligibility Adjudication Committee will be required prior to randomization (Day 1/Visit 2).

Note: The classification criteria can be met with past signs, symptoms, and diagnostic findings (e.g., the muscle biopsy findings can be from a past procedure).
3. Participants meeting both of the following disease severity criteria (at screening and randomization [Day 1/Visit 2]):
   MMT-8 score ≥80 and ≤142 (out of 150 total possible) and
   Active cutaneous manifestation of dermatomyositis documented as CDASI Activity Score ≥6.

Note: For MMT-8, if a non-assessable muscle is present, the score will be automatically adjusted in the electronic case report form [eCRF], and a total score out of 150 will be provided.
4. For participants with onset of dermatomyositis symptoms within 3 years prior to screening, have a documented computed tomography (CT) (or positron emission tomography-computed tomography [PET-CT]) scan with contrast of the chest, abdomen, and pelvis, taken after the onset of symptoms and within 1 year prior to screening, without findings suggestive of malignancy.

Note: For participants without available CT (or PET-CT) scan results, a CT (or PET-CT) scan may be performed during the Screening Period.
5. Current therapy consisting of corticosteroid≤20 mg/day (including a dose of 0 mg [i.e., not taking corticosteroid]) of prednisone or at screening. Total duration of prescribed corticosteroid therapy should be at least 12 weeks prior to randomization (Day 1/Visit 2), where the dose must be stable for at least 4 weeks prior to randomization.
6. At most, one systemic non-steroid immunomodulatory/immunosuppressive therapy, with a stable dose for at least 12 weeks prior to randomization (Day 1/Visit 2).

Note: An antimalarial is allowed in addition to a non-steroid immunomodulatory/immunosuppressive therapy, and the antimalarial dose must be stable for at least 12 weeks prior to randomization (Day 1/Visit 2).

Note: Participants not receiving either corticosteroids or non-steroid immunomodulatory/immunosuppressive therapy for the treatment of dermatomyositis require documented failure of response (which may include medical history by participant report) or intolerance to at least 1 prior dermatomyositis-related corticosteroid and/or non-steroid immunomodulatory/immunosuppressive therapy (an antimalarial alone is not sufficient to meet this criterion) and must have been discontinued prior to randomization (Day 1/Visit 2).
7. Participants with disease activity that includes abnormalities in at least 2 of the 5 following International Myositis Assessment and Clinical Studies Group (IMACS) Disease Activity CSMs (at least 2 must be present at both screening and randomization (Day 1/Visit 2), however the 2 CSMs do not need to be the same ones at both timepoints):
i. PhGA-VAS ≥2 cm,
ii. PtGA-VAS of ≥2 cm,
iii. HAQ Disability Index ≥0.25,
iv. At least one muscle enzyme (creatine kinase [CK], aldolase, alanine aminotransferase [ALT], aspartate aminotransferase [AST], and lactate dehydrogenase [LDH])>1.5 times upper limit of normal (ULN) at screening,
v. Extramuscular Global Assessment-VAS ≥2 cm.

### Weight.

8. Participants weighing >40 kg to <130 kg, and with a body mass index (BMI)<40 kg/m2.

Open-Label Extension Period. Participants who complete the Blinded Treatment Period and meet the eligibility requirements may enter the OLE Period and receive open-label Brepocitinib 30 mg QD. The OLE Period begins immediately following completion of assessments at Week 52/Visit 12 and ends at Week 104/Visit 17. During the OLE Period, a participant may receive rescue therapy in addition to allowed concomitant dermatomyositis medication.

Off-Drug Follow-Up Period. To assess safety over the 4-week Off-Drug Follow-Up Period, participants will return to the clinic for a Follow-Up Visit (at Week 56 for participants who complete the Blinded Treatment Period but do not enter the OLE Period; at Week 108 for participants who complete the OLE Period; or 28 days after the last dose of study drug for participants who withdraw from the study early during either the Blinded Treatment Period or OLE Period).

Duration of Treatment. The total duration of treatment will be up to 104 weeks (52 weeks of study drug [active or placebo] taken during the Blinded Treatment Period and 52 weeks of active study drug taken during the OLE Period).

Data Monitoring/Other Committees. This study will include the following external independent committees: an Independent Eligibility Adjudication Committee to provide verification of eligibility of disease diagnosis (for potential participants with probable idiopathic inflammatory myopathy); an Independent Data Monitoring Committee to monitor safety of participants; and a Clinical Event Classification Committee to adjudicate cardiovascular, thromboembolic, and malignancy events.

### Example 3

Solubility: Brepocitinib is a highly soluble molecule across the physiological pH range and is fully soluble in 250 mL of the media across this pH range.

Permeability: Brepocitinib is also a highly permeable compound as evidenced from a human mass balance study following a single oral administration of 60 mg (300 nCi) C-Brepocitinib in which the total recovery of the orally administered radioactive dose over a period of 192 hours post dose was 96.7%±6.3%, with 88.0%±8.0% in the urine and 8.7%±2.1% in the feces.

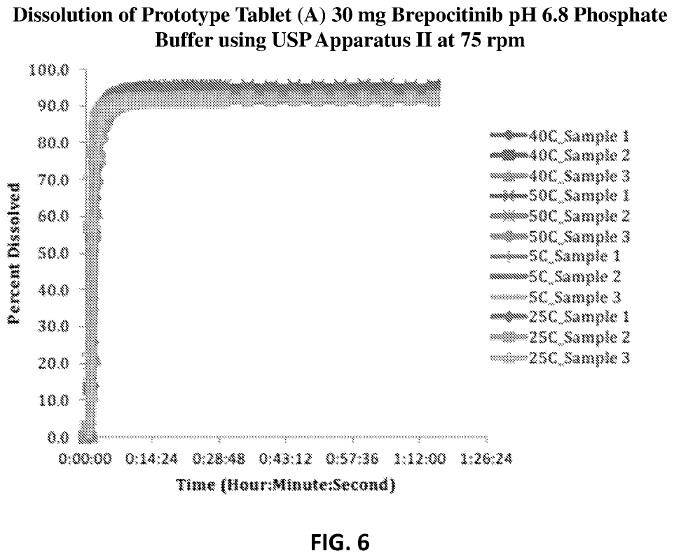

Dissolution: Brepocitinib demonstrates rapid dissolution from immediate-release (IR) tablets (>85% in 30 minutes). An illustrative dissolution profile for a prototype 30 mg tablet in pH 6.8 phosphate buffer using USP Apparatus II, 75 rpm is shown in FIG. 6. As the solubility of Brepocitinib is similar across the physiological pH range, the dissolution is expected to be rapid in pH 1.0, and pH 4.5 as well. The final to-be-marketed product will be tested in all three-pH media for confirmation of rapid dissolution. The to-be-marketed product will be developed at a commercial manufacturing site that is to be decided and will be different from that used to manufacture the current clinical trial material.

Pharmacology. Brepocitinib inhibits various TYK2- and JAK1-dependent functions in different cell types such as cytokine-induced phosphorylation of signal transducer and activator transcriptions (STATs) in human peripheral blood mononuclear cells (PBMCs) and whole blood; differentiation of T helper (Th) 1, Th17, and B cells, interleukin (IL)-12-induced IFNγ production in human PBMCs; and IFN-1 gene signature induced by immune complexes in human PBMCs. The anti-inflammatory effects by Brepocitinib have also been demonstrated in vivo using a rat adjuvant-induced arthritis disease model.

Absorption, Distribution, Metabolism, and Excretion. The ADME profiles of Brepocitinib and major circulating metabolite M1 have been studied in various in vitro and in vivo studies. The pharmacologically inactive M1 is not unique to humans and is well represented in the plasma of rats based on exposures in toxicity studies. The clearance pathways for Brepocitinib in humans primarily involve cytochrome P450 (CYP) metabolism, predominantly via CYP3A4, and to a lesser extent, CYP1A2. MI is primarily eliminated by urinary excretion.

Brepocitinib has a relatively low volume of distribution (60 L in adult study participants) and is a substrate of P-glycoprotein (P-gp; multidrug resistance protein 1 [MDR1] or adenosine triphosphate (ATP) binding cassette subfamily B member 1 [ABCB1]), therefore penetration across the blood-brain barrier is expected to be low. After oral administration of Brepocitinib to humans, >95% of the dose was recovered over an 8-day period, indicating little potential for long-term retention of drug-related material. Brepocitinib is not highly bound to plasma proteins (unbound fraction of 0.609 in man).

Based on in vitro profiling, there is low potential for Brepocitinib to be a perpetrator of metabolic drug-drug interactions through direct inhibition and induction mechanisms. In vitro inhibition of the drug transporters P-gp, breast cancer resistance protein (BCRP), multidrug and toxin extrusion (MATE) 1, organic cation transporter (OCT) 1, and OCT2 was also observed. The M1 metabolite was also an inhibitor of MATE2K.

Toxicology. The in vitro profile of Brepocitinib in functional assays, ligand binding, or enzyme activity in a broad panel of receptors, kinases, enzymes, ion channels, transporters, and phosphodiesterase subtypes indicates low potential for off-target pharmacology.

Nonclinical toxicity studies conducted to support the clinical use of Brepocitinib by the oral route of administration include Good Laboratory Practice (GLP) single- and repeat-dose oral general toxicity studies of up to 6-months duration in rats and up to 9-months duration in cynomolgus monkeys; in vitro and in vivo genotoxicity studies; carcinogenicity studies of 2-years duration in rats and 6-months duration in transgenic (Tg) mice; reproductive and developmental toxicity studies (fertility and embryo fetal development) in rats and rabbits; a juvenile toxicity study in rats; and a phototoxicity study in pigmented rats. Chronic, oral toxicology studies in rats and monkeys have 54- and 7.8-fold area under the concentration-time curve (AUC) exposure margins, respectively, from the end of study exposure at the no observed adverse effect level (NOAEL) to the clinical exposure of Brepocitinib 30 mg QD. The toxicology findings were consistent with the mechanism of action, with changes to the immune and hematolymphopoietic systems and target tissues including thymus, spleen, lymph nodes, and bone marrow. In cardiovascular safety pharmacology studies following oral administration, Brepocitinib-related effects on blood pressure, heartrate, and corrected QT interval (QTc) interval were observed.

Immunotoxicity and phototoxicity studies have also been conducted. Carcinogenicity studies in Tg mice (6 month) and rats (2 year) have been completed. No Brepocitinib-related neoplastic findings were observed in the 6-month mouse study. Brepocitinib-related findings consistent with the mechanism of action (lower thymus weight and cellularity, infection) or not relevant to humans (exacerbation of chronic progressive nephropathy in the kidney) were also observed. Fertility studies in rats have been completed. No adverse effects were observed relative to male rat fertility at the highest dose tested (55 mg/kg/day, >50-fold margin to clinical exposures

Absorption. Brepocitinib area under the concentration-time curve from time 0 extrapolated to infinity (AUC (0-∞)) and Cmax increase in an approximately dose-proportional fashion from 1 to 100 mg. Brepocitinib is absorbed rapidly after oral administration with median time to maximum concentration (Tmax) values generally <1.5 hours after single- and multiple-dose administration. The absolute bioavailability of a Brepocitinib solution is approximately 75%, and the bioavailability of Brepocitinib administered as tablets relative to administration as a solution was approximately 96%. Furthermore, the fraction absorbed of Brepocitinib after administration as a solution was approximately 100%. These results demonstrate that Brepocitinib is well-absorbed after oral administration and is a high-permeability compound according to the BCS criteria. Administration of Brepocitinib with food (high-fat meal) delays the oral absorption of Brepocitinib but does not have a clinically relevant effect on the extent of absorption relative to administration in the fasted state. Therefore, Brepocitinib can be administered without regard to meals.

Distribution. Brepocitinib is not highly bound to plasma proteins. Brepocitinib fraction of unbound drug was 0.609 at 2 μM in human plasma. Brepocitinib volume of distribution at steady state (Vss) was 60.0 L after a single intravenous dose, indicating distribution outside of plasma.

Metabolism. Brepocitinib is metabolized extensively after oral administration. The in vitro fraction metabolized was 0.77 and 0.14 for CYP3A4/5 and CYP1A2. Approximately 80% of an orally administered, radiolabeled dose was recovered as Brepocitinib metabolites in the urine. The inactive M1 metabolite is the primary metabolite observed in plasma (37.1% of Brepocitinib-related material) and urine (58.4% of Brepocitinib-related material) in the 12-hour post dose period. After a single dose of Brepocitinib 30 mg, the mean (% coefficient of variation [CV]) M1 -to-Brepocitinib ratios were 1.65 (126) and 0.849 (109) for AUC (0-∞) and Cmax, respectively. Inhibition of Brepocitinib metabolism by itraconazole (a strong CYP3A and P-gp inhibitor) decreased the mean (% CV) metabolite-to-parent ratios to 1.22 (115) and 0.604 (101) for AUC (0-∞) and Cmax, respectively.

Elimination. Brepocitinib elimination half-life (t½) generally ranged from 4 to 8 hours after single dose administration. Steady state was reached by Day 8 of QD administration and accumulation in the plasma was <50% upon repeated single daily doses of up to 100 mg. Renal excretion is a minor route of elimination for Brepocitinib. Approximately 8% of a radiolabeled dose was recovered as unchanged Brepocitinib in the urine after a single 60 mg dose, and ≤16% of the dose was recovered in urine after repeated daily doses of 10 to 175 mg. Less than 10% of a radiolabeled Brepocitinib dose was recovered in the feces. The M1 metabolite is the major excreted metabolite, accounting for 52.1% of the total dose excreted in urine and feces.

### Example 3

### Brepocitinib Prevents Type I Interferon Induced Damage in Cultured Myocytes and Endothelial Cells

Human skeletal muscle myoblasts (Lonza, CC-2580) were cultured in SkGM™-2 media (Lonza, CC-3245) and sub-cultured using ReagentPack™ Subculture Reagents (Lonza, CC-5034). They were then differentiated into myotubes by culturing in differentiation medium [DMEM/F12+2% Horse serum (Thermofisher, 16050122)] for 3-5 days until most of the myoblast cells differentiated into multinucleated myotubes. The myotubes were then preincubated with vehicle or Brepocitinib prior to type I interferon (R&D, 11200-2) exposure at concentrations (FIG. 7). After 48 hours of treatment, cells were fixed and stained with Myosin 4 Monoclonal antibody (MF20; Thermofisher, 14-6503-82) and Hoechst for nuclei visualization. The effect on total myotube surface area was evaluated via fluorescent image analysis. Similarly, human dermal microvascular endothelial cells (HMEC-1; ATTC, CRL-3243) were cultured in complete growth medium [MCDB-131 (Thermofisher, 10372019)+10% FBS (ATCC, 30-2020)+10 ng/mL EGF (Thermofisher, PHG0314)+1 μg/mL hydrocortisone (StemCell, 74142)+2 mM L-glutamine (Thermofisher, 25030081)] and sub-cultured using trypsin+0.25% EDTA. HMECs were then cultured on Matrigel Growth Factor Reduced Basement Membrane Matrix-coated plates (250 μL/well in 24-well plates; Corning, 356231) and allowed to form vascular networks. Subsequently, the HMEC's were pretreated with Brepocitinib prior to type I IFN exposure at the same concentrations as for myotubes. The effect of type I IFN exposure±Brepocitinib treatment on the number of nodes, master segment length, and total mesh area was evaluated by taking bright-field images every 16 hours.

Type I IFN induced damage was almost completely prevented by Brepocitinib preincubation in both myotubes and endothelial cells at a clinically relevant concentration equivalent to the average unbound plasma concentration after 30 mg QD (FIG. 7). These biological studies are supportive of the potential of a TYK2/JAK1 inhibitor to treat both skin and muscle inflammation in DM.

### Example 4

### Brepocitinib Clinical Development History

As part of the clinical development program for oral Brepocitinib, a number of Phase 1 and Phase 2 studies have been completed or have a report in preparation. The Phase 2 studies evaluated the safety, tolerability, and efficacy across a wide range of Brepocitinib doses for the treatment of plaque psoriasis, psoriatic arthritis, alopecia areata, Crohn's disease, ulcerative colitis, hidradenitis suppurativa, systemic lupus erythematous (SLE), and nonsegmental vitiligo, and have demonstrated statistically significant and/or clinically relevant efficacy. The multiple autoimmune diseases for which there is observed clinical efficacy data for Brepocitinib have mechanistic pathway similarities to DM. For example, psoriasis is driven by IL-17, which is downstream of IL-12 and IL-23 signaling via TYK2 and/or JAK1, and alopecia areata is characterized by excessive activity of IFN-γ, a type II interferon which also signals via JAK1. Of note, dose dependent reductions in IP-10 (CXCL10), an IFN-γ inducible gene, have been observed in Brepocitinib clinical studies, separating from placebo at doses ≥10 mg QD. Importantly, in these phase 2 studies, clinical response with Brepocitinib treatment rapidly separates from placebo. For example, in the phase 2 study assessing Brepocitinib's efficacy in psoriatic arthritis, Brepocitinib 30 mg achieved a 52% ACR20 response rate after 4 weeks compared to a 19% response rate in placebo patients. This suggests Brepocitinib has a rapid onset of action that can begin alleviating patient symptoms quickly.

Brepocitinib has been generally well-tolerated in studies to date. Brepocitinib is being evaluated in a double-blind, randomized, placebo-controlled Phase 3 study in DM (NCT0543726; VALOR Study). With a planned enrollment of 225 subjects, the VALOR Study is the largest well-controlled trial in DM to date. Adult patients with definite or probable idiopathic inflammatory myopathy (IIM), who meet the subclassification criteria for DM, have both muscle and skin disease involvement (classic DM), and are receiving (or are not responsive to or are intolerant to) corticosteroids and/or immunomodulatory/immunosuppressive therapies are eligible to enroll. The primary endpoint is the total improvement score (TIS) at week 52. The TIS is a composite endpoint based on the following six CSM scores: the Physician Global Disease Activity, the Patient Global Disease Activity, the Health Assessment Questionnaire, MMT-8, Extramuscular Disease Activity, and muscle enzymes. To evaluate the steroid-sparing effect of Brepocitinib, participants using glucocorticoids at baseline must taper to an oral glucocorticoid dose of prednisone≤5 mg/day (or equivalent) by week 36. In sum, clinical and molecular data support the use of Brepocitinib, an oral TYK2/JAK1 inhibitor, as a rational and targeted approach for the treatment of DM, with an ongoing double-blind, randomized, placebo-controlled Phase 3 trial to confirm this hypothesis.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A method for treating Dermatomyositis (DM), the method comprising administering to a subject in need of treatment thereof an effective amount [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone (Brepocitinib) 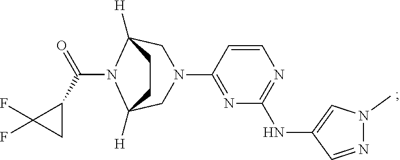 [SMILES: Cn1cc(Nc2nccc(N3C[C@H]4CC[C@@H](C3)N4C(=O)[C@@H]3CC3(F)F)n2)cn1]
or a pharmaceutically acceptable salt thereof, wherein the compound or pharmaceutically acceptable salt thereof is administered in a dosage of about 10 mg to about 60 mg.

2. The method of claim 1, wherein the administration of an effective amount of Brepocitinib to the subject has one or more effects selected from the group consisting of:
a. decreases or inhibits IFN-I activity in the subject;
b. decreases or inhibits cytokine signaling dysregulation implicated in DM;
c. prevents or reduces damage in human myocytes and microvasculature; and
d. interferes with DM life cycle.

3. The method of claim 1 wherein the pharmaceutically acceptable salt is p-toluenesulfonic acid salt.

4. The method of claim 1, wherein the compound is administered orally.

5. The method of claim 1, wherein the compound is administered in a dosage of about 30 mg.

6. The method of claim 1, wherein the compound is administered in a dosage of about 15 mg.

7. The method of claim 1, wherein the compound administered is taken daily.

8. The method of claim 1, wherein the compound administered is in divided doses administered two, three or four times per day.

9. The method of claim 1, wherein the compound is administered for less than or about 104 weeks.

10. The method of claim 1, wherein the compound is administered for more than or about 4 weeks.

11. The method of any claim 1 wherein the subject is a mammal.

12. The method of claim 11 wherein said mammal is a human.

13. The method of claim 11 wherein said mammal is a canine.

14. Use of Brepocitinib to enable steroid tapering of claim 1 for the manufacture of a medicament for the treatment of Dermatomyositis in a subject in need thereof.
